(19) United States
(12) Patent Application Publication
(10) Pub. No.: US 2006/0063859 A1
(43) Pub. Date: Mar. 23, 2006

(54) Transition metal-catalyzed synthesis of dendritic polymers

(76) Inventor: Zhibin Guan, Irvine, CA (US)

Correspondence Address: ROBERT D. FISH;RUTAN & TUCKER LLP, 611 ANTON BLVD 14TH FLOOR, COSTA MESA, CA, 92626-1931, US

(21) Appl. No.: 11/210,347

(22) Filed: Aug. 23, 2005

Related U.S. Application Data
(60) Provisional application No. 60/611,044, filed on Sep. 17, 2004.

(51) Int. Cl. A61K 47/48; C09D 151/00

(52) U.S. Cl. 523/201; 525/54.1

(57) ABSTRACT

Dendritic amphiphilic polymers are contemplated. Most preferably, such polymers will be fabricated in a single step to the final product that may further be derivatized with, among others, biological relevant molecules. In alternative aspects, precursors of such molecules are prepared in a single step, and the precursors are then reacted to the dendritic amphiphilic polymers.

This application claims the benefit of our provisional patent application with the Ser. No. 60/611,044, which was filed Sep. 17, 2004, and which is incorporated by reference herein.

This invention was made with government support under grant number NSF DMR-0135233 by the National Science Foundation and grant number DAAD19-01-1-0686 by the Army Research Office. The government may have certain rights in the invention.

### FIELD OF THE INVENTION

The field of the invention is amphiphilic polymers, and especially dendritic amphiphilic polymers and methods therefor.

### BACKGROUND OF THE INVENTION

Amphiphilic compounds are of general interest in numerous disciplines in life sciences, especially in pharmacology and general chemistry, and numerous amphiphilic compounds are known in the art. However, traditional amphiphilic compounds usually forms micelles or aggregates that are often not stable, or tend to interfere with the integrity of reagents that are mixed with such compounds. Moreover, many of the well known amphiphilic compounds have further disadvantages, including limited possibilities for functionalization, and relatively small size.

Thus, for these and various other reasons, new amphiphilic compounds, and especially amphiphilic dendritic compounds have been developed with remarkable properties. For example, self-organizing amphiphilic dipeptide dendritic compounds were described that form pores in membranes (see e.g., Nature. 2004 Aug. 12; 430(7001):764-8), or that arrange into cylinders where poly(para-phenylene) was used with hydrophobic and hydrophilic dendrons (see e.g., Angewandte Chemie International Edition 1999; Vol. 38(16):2370-2372). While such compounds are conceptually highly interesting, their usefulness is often limited due to the high price of manufacture, instability, or immunogenicity.

Other known amphiphilic dendritic compounds are fabricated from branched monomers with A-BB type structure to form regularly shaped dendritic compounds that can then be easily derivatized in a subsequent step to an amphiphilic dendritic compound. Such compounds are often highly predictable in terms of molecular weight, general structure, and reactivity and often provide compounds with well-defined molecular weight. Moreover, synthesis is typically simple. However, certain disadvantages nevertheless remain, including a limited range of compatible reactive groups that will lead to satisfactory dendritic compounds. Additionally, multiple reactions have to be carried out to increase molecular weight of the desired compounds. Still further, many of such dendritic compounds will have a hydrophilic core and a hydrophobic shell, which is often undesirable for biological applications.

Further known amphiphilic dendritic compounds include poly(2-methyl-2-oxazoline)-block-poly(amido amine) dendromers (see e.g., Macromolecular Rapid Communications 2003, Vol. 18(10):945-952), and poly(propylene oxide)-polyglycerol copolymers (Tetrahedron 59 (2003):4017-4024). While such compounds will provide at least some advantages over other known dendritic amphiphilic compounds, they are often difficult to synthesize. Moreover, at least some of such compounds tend to be incompatible or problematic in biological applications.

Therefore, while there are numerous compositions and methods for dendritic polymers are known in the art, all or almost all of them suffer from one or more difficulties. Thus, there is still a need for new dendritic polymers, and especially amphiphilic dendritic polymers.

### SUMMARY OF THE INVENTION

The present invention is directed to amphiphilic dendritic polymers that can be prepared in a simple manner. Among other advantages, contemplated synthetic methods allow preparation of dendritic polymers over a wide variety of chemical compositions and molecular weight, and also allow subsequent derivatization.

In one aspect of the inventive subject matter, an amphiphilic core-shell copolymer has a core with a plurality of branches, wherein the branches have unequal distances between at least two branch points, wherein the core comprises a first polymer, wherein the copolymer further comprises a shell that comprises a second polymer, and wherein the second polymer is covalently coupled to a terminus of a branch of the first polymer.

Most preferably, the first polymer has a dendritic structure, and/or is hydrophobic or fluorophobic, while the shell polymer is hydrophilic or fluorophobic. Thus, suitable first polymers may comprise a polyolefin and second polymers may comprise a polyethylene glycol or a fluorocarbon oligomer or polymer. Where desirable, the second polymer may further comprise a reactive group that is suitable for derivatization with a biological molecule.

Therefore, in another aspect of the inventive subject matter, a reaction mixture comprises a plurality of first-monomers and second monomers, and a polymerization catalyst capable of a chain walking reaction, wherein the second monomer is functionalized with a group such that (a) the second monomer is hydrophilic, or that (b) the group is suitable for reaction with a hydrophilic reagent. In such mixtures, it is generally preferred that the first and second monomers comprise an optionally substituted ethylene group. For example, preferred first monomers may comprise an α-olefin, and second monomers may comprise 2,2-dimethyl-pent-4-enyl-epoxide, an optionally protected 2,2-dimethyl-pent-4-enyl-alcohol, and/or or an optionally protected 2,2-dimethyl-pent-4-enyl-acid. it is further typically preferred that the polymerization catalyst comprises an organometallic catalyst, and most preferably includes a late transition metal in complex with at least one coordinating atom.

Consequently, a method of forming a dendritic amphiphilic polymer is contemplated in which a plurality of first monomers and a plurality of second monomers are provided, wherein at least some of the second monomers include a hydrophilic group or a group suitable for reaction with a hydrophilic reagent. In another step, the first and second monomers are reacted under conditions that promote (a) formation of a branched polymer, and (b) covalent bonding of the second monomers to termini of branches of the branched polymer.

Preferably, the dendritic amphiphilic polymer in such methods has a hydrophobic core and a hydrophilic shell. Therefore, and among other choices, suitable first monomers have a structure of R₁R₂C═CR₃R₄, wherein R₁, R₂, R₃, and R₄ are independently hydrogen, halogen, and optionally substituted lower alkyl, and suitable second monomers have a structure of R₁R₂C═CR₃R₅, wherein R₁, R₂, and R₃, are independently hydrogen, halogen, and optionally substituted lower alkyl, and wherein and R₅ is or comprises a polar group. Polar groups that are especially preferred include a hydroxy group, a carboxy group, an epoxy group, a substituted ester, a substituted amide, a substituted imide, a polyol, and a polyether. Alternatively, the group suitable for reaction with the hydrophilic reagent is an alcohol, an acid, or an epoxy group. Such methods will further include a step of reacting the alcohol, acid, or epoxy group with a polar reagent. Most preferably, the step of reacting comprises a step of chain walking polymerization, and is typically performed at a pressure of less than 0.5 atm of the first monomer.

Various objects, features, aspects and advantages of the present invention will become more apparent from the following detailed description of preferred embodiments of the invention, along with the accompanying drawing.

### BRIEF DESCRIPTION OF THE DRAWING

FIG. 1 is a schematic exemplary representation of a one-step synthesis of a dendritic amphiphilic copolymer according to the inventive subject matter.

FIG. 2A is a fluorescence emission spectrum (λex=570 nm) of Nile Red with addition of copolymer 1 at different concentration (λmax=626 nm).

FIG. 2B is a fluorescence emission spectrum (λex=570 nm) of Nile Red as a function of concentration of copolymer 1 (▪), copolymer 2(▴), and SDS (●), respectively.

FIG. 3 is a graph depicting the concentration of encapsulated Nile Red plotted against the concentration of copolymer 1 (▪) and copolymer 2 (▴).

FIG. 4 is a graph depicting the number-of-dye molecules per polymer molecule at different concentrations of copolymer 1 (▪) and copolymer 2 (▴).

FIG. 5A is a schematic depicting maximum absorbance in UV/VIS spectra of Nile Red dispersion (30 μM) followed by the addition of SDS at different concentrations.

FIG. 5B is a schematic depicting maximum absorbance in UV/VIS spetra of different concentrations of Nile Red aqueous solution with SDS at a concentration of 30 mg/mL.

FIG. 6A is a UV/VIS spectrum of Nile Red in water with different concentrations of Copolymer 1.

FIG. 6B is a UV/VIS spectrum of Nile Red in water with different concentrations of Copolymer 2.

FIG. 6C is a UV/VIS spectrum of Nile Red in water with different concentrations of SDS.

### DETAILED DESCRIPTION

The inventor has discovered that dendritic amphiphilic polymers can be prepared in a simple reaction in which a plurality of first and second monomers are copolymerized under conditions that allow formation of a branched core polymer onto which, in the same reaction or in a subsequent reaction, the shell polymer is covalently coupled. Most preferably, the shell polymer is covalently coupled to the ends of the branches of the core polymer. In further contemplated aspects, the reaction performed is a chain walking polymerization using an organometallic catalyst.

As used herein, the term “amphiphilic” in conjunction with a molecule denotes that the molecule contains (among optionally other groups) both at least one hydrophobic and at least one hydrophilic group. As also used herein, the term “core-shell copolymer” refers to a copolymer that has a conformation in which one portion (typically a hydrophilic or hydrophobic portion) is at least partially surrounded by another portion or plurality of portions (typically a hydrophilic or hydrophobic portion with opposite philicity/phobicity of the core). Thus, the term “shell” as used herein refers to one or more monomers that are reacted to the core to thereby form the copolymer, and the term “core” refers to a polymer (typically branched, most typically dendritic) to which the shell is covalently coupled. As still further used herein, the term “fluorophobic” refers to a decreased or lacking propensity of a compound to solubilize in a fluorous hydrocarbon, while the term “fluorophilic” refers to an increased propensity of a compound to solubilize in a fluorous hydrocarbon.

As further used herein, the term “branch point” refers to a point in a polymer in which one polymer backbone (comprising a plurality of monomers) is covalently coupled to another polymer backbone (comprising a plurality of monomers). As also used herein, the term “dendritic” refers to a branched polymer in which one branch of a polymeric backbone has at least one other branch.

The term “chain walking” as still further used herein refers to a mechanism in which a catalyst proceeds along a hydrocarbon chain in a manner as depicted in Scheme 1 below. Further aspects of contemplated chain walking are described by Guan, Z.; Cotts, P. M.; McCord, E.; and McLain, in J. Science 1999, 283, 2059, which is incorporated by reference herein.

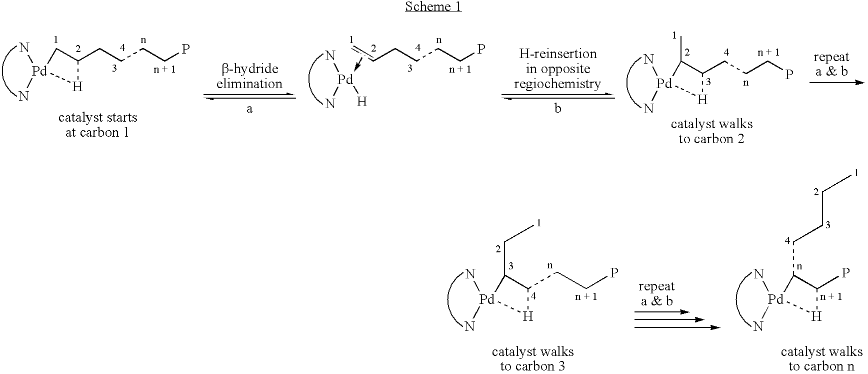

Using the above depicted mechanism, the inventor discovered that branched polymeric molecules can be manufactures, and especially those that are prepared from optionally substituted alpha-olefins. Remarkably, and as further discussed in more detail, the degree of branching in such polymeric synthesis can be controlled by control of the pressure or concentration for the monomers. An exemplary synthesis of branched polyethylene is depicted in Scheme 2 below in which low pressure ethylene conditions favor branched and dendritic polyethylene while high pressure ethylene conditions favor a linear topology (see e.g., Guan, Z.; Cotts, P. M.; McCord, E.; and McLain, in J. Science 1999, 283, 2059 and Chen, G.; Ma, S. X. and Guan, Zhibin. J. Am. Chem. Soc. 2003, 125, 6697).

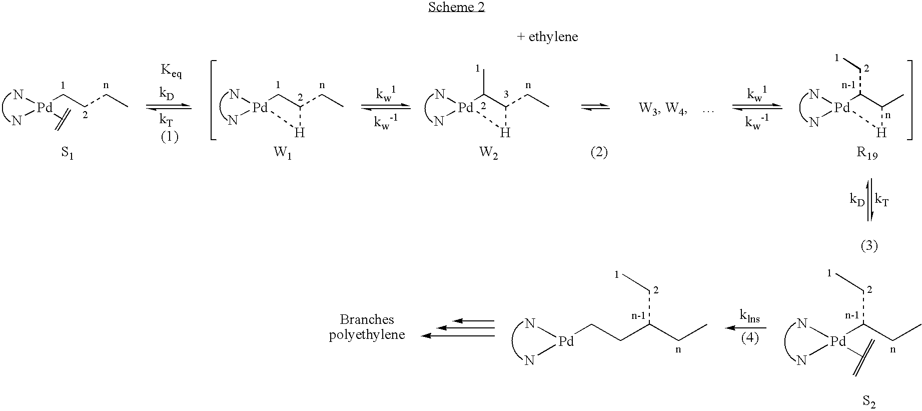

Using the above chemistry, the inventor has now discovered that where a second type of monomer, and especially an optionally substituted alpha-olefinic monomer is employed, functionalized dendritic polymers can be prepared in a reaction as exemplarily depicted in Scheme 3A below (Representative (but not limiting) conditions for such reactions are described in the exemplary section below and in Chen, G.; Guan, Zhibin. J. Am. Chem. Soc. 2004, 126, 2662). It should be particularly noted that insertion of substituted olefins occurs only when Pd walks to a primary carbon, that is, a chain end (Gottfried, A. C.; Brookhart, M. Macromolecules 2003, 36, 3085-3100.). Thus, copolymerization of ethylene and an PEG containing olefin at low ethylene pressure using such synthetic strategy will produce a dendritic copolymer with a hydrophobic PE core and a hydrophilic PEG shell.

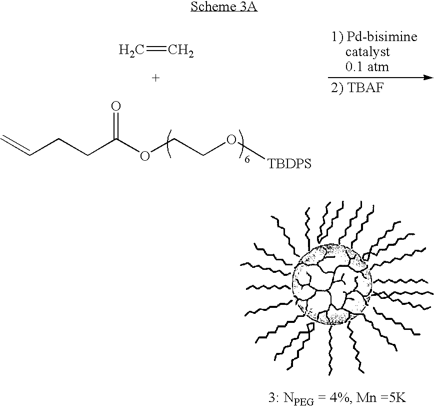

Of course, it should be recognized that the choice of the substituted alpha-olefinic monomer need not be limited to the monomer of Scheme 3A, but numerous other monomers are also deemed suitable so long as such monomers will participate in the polymerization reaction as described above. Consequently, in one aspect of the inventive subject matter, suitable monomers will include a terminal double bond and at least one substituent that has a group or groups with desired functionality and/or polarity. For example, where a shell should include hydrophilic carbohydrate moieties, a reaction according to exemplary Scheme 3B may be employed.

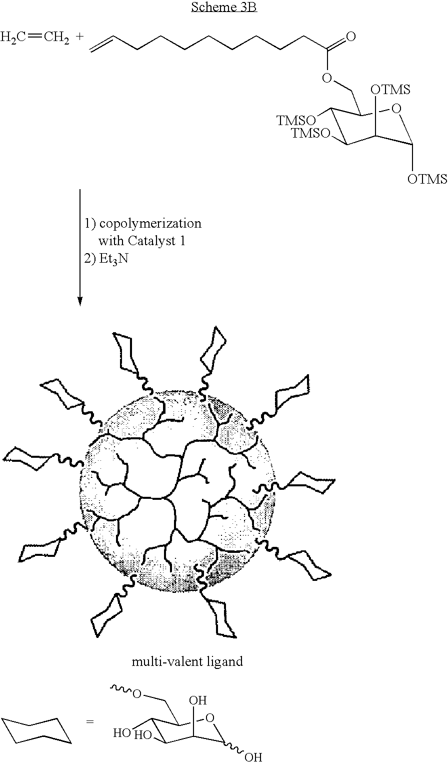

On the other hand, where it is desired that the shell is fluorophilic/hydrophobic, the monomers can be chosen according to Scheme 3C below in which the substituted alpha-olefinic compound includes a (per)fluorinated portion.

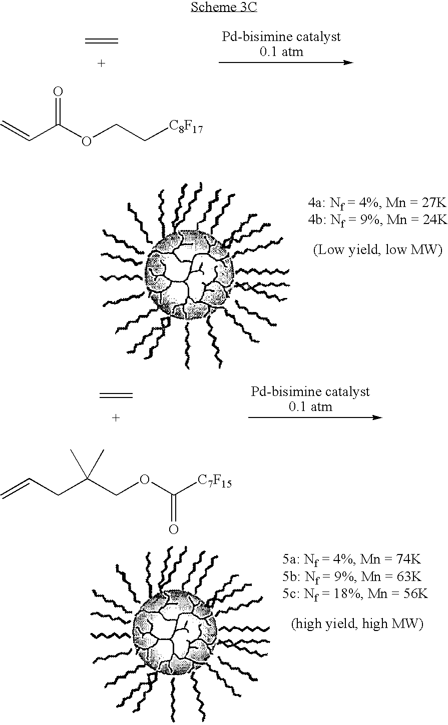

Where a suitable monomer can not be prepared in a convenient or cost-effective manner, or is otherwise not available, it should be recognized that the copolymer may also be prepared using a modified second monomer that provides only a reactive group to which then—in a separate reaction—a suitable moiety can be coupled. An exemplary reaction sequence and suitable modified monomers are Scheme 4 below.

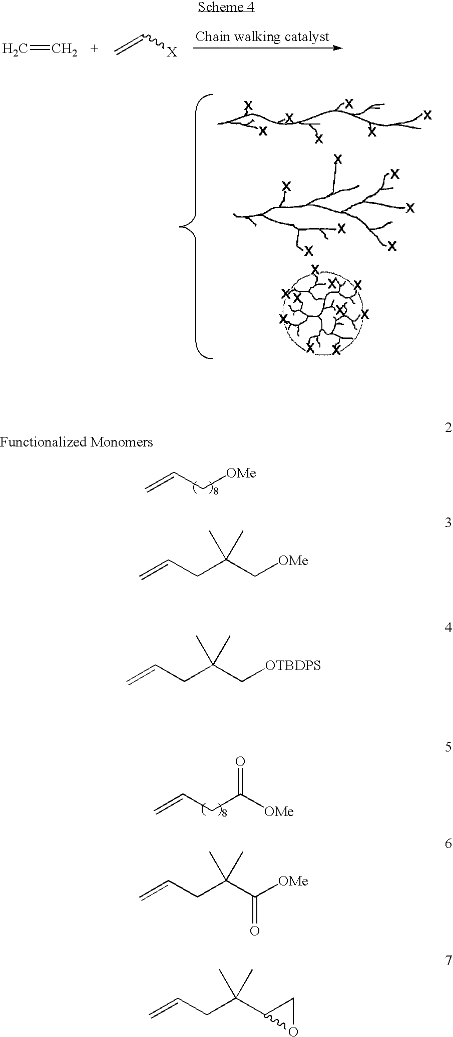

Consequently, it should be recognized that the first and second monomers can be selected such that the monomers participate in a polymerization in which a branched and/or dendritic copolymer is formed. While not limiting to the inventive subject matter, it is therefore preferred that the monomers include an ethylene group, and that the reaction has an organometallic catalyst to enable a chain walking reaction. For example, preferred first monomers include those having a structure of R₁R₂C═CR₃R₄, wherein R₁, R₂, R₃, and R₄ are independently hydrogen, halogen, and optionally substituted lower alkyl or aryl (i.e., C1-C7, linear, branched, or cyclic). With respect to the second and optionally modified monomer, it is contemplated that the monomer may either include a desired functional portion (e.g., hydrophilic or hydrophobic polymer, one or more reactive groups, optionally protected, one or more carbohydrate moieties, etc.) or an optionally protected functional group that can react with a desired substituent (e.g., hydrophilic reagent, polypeptide, linker, lipid, etc.). Therefore, suitable modified monomers may have a structure of R₁R₂C═CR₃R₅, wherein R₁, R₂, and R₃, are independently hydrogen, halogen, and optionally substituted lower alkyl, and wherein R₅ is a polar (e.g., hydroxy group, carboxy group, epoxy group, substituted ester, substituted amide, substituted imide, polyol, and/or polyether) or a reactive (optionally protected) group. Thus suitable modified monomers include 2,2-dimethyl-pent-4-enyl-epoxide, an optionally protected 2,2-dimethyl-pent-4-enyl-alcohol, and an optionally protected 2,2-dimethyl-pent-4-enyl-acid. Such reactive groups may then be reacted with all reagents known in the art, and especially include nucleic acids, polypeptides, and carbohydrates. Further especially preferred reagents include polar reagents (e.g., polyols, saccharides, hydrophilic peptides, etc.).

Therefore, the inventor especially contemplates an amphiphilic core-shell copolymer that has a core with a plurality of branches, wherein the branches have unequal distances between at least two branch points, wherein the core comprises a first polymer, wherein the copolymer further comprises a shell that comprises a second polymer, and wherein the second polymer is covalently coupled to a terminus of a branch of the first polymer. Alternatively, or additionally, the core may also be functionalized with a shell monomers, wherein at least some of the shell monomers include a reactive group that may be further reacted with a biologically relevant molecule (e.g., nucleic acid, peptide, saccharide, etc). On the other hand, and especially where the core is hydrophobic and the shell is hydrophilic, the shell polymer (e.g., PEG) may include a terminal reactive group (e.g., maleimide, amino group, etc.) that may further be reacted with a biologically relevant molecule. Thus, and viewed from another perspective, a reaction mixture may comprise a plurality of first monomers and second monomers, and a polymerization catalyst capable of a chain walking reaction, wherein the second monomer is functionalized with a group such that (a) the second monomer is hydrophilic, or that (b) the group is suitable for reaction with a hydrophilic reagent.

With respect to the catalyst, it is generally contemplated that the catalysts will promote copolymer formation, and especially branched and/or dendritic copolymer formation. Therefore, particularly suitable catalysts include those that catalyze a chain walking reaction. For example, such catalysts will include organometallic catalysts, and particularly those in which a late transition metal is in complex with at least one coordinating atom. Among other suitable choices, preferred catalysts include those according to Formula 1 below.

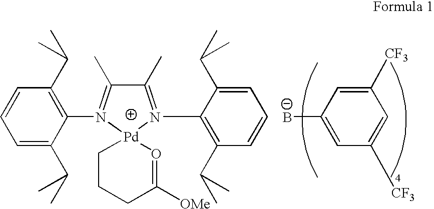

Therefore, it should be recognized that a method of forming a dendritic amphiphilic polymer will preferably include a step in which a plurality of first monomers and a plurality of second monomers are provided, wherein at least some of the second monomers include a hydrophilic group or a group suitable for reaction with a hydrophilic reagent. In another step, the first and second monomers are reacted under conditions that promote (a) formation of a branched polymer, and (b) covalent bonding of the second monomers to termini of branches of the branched polymer.

Experiments

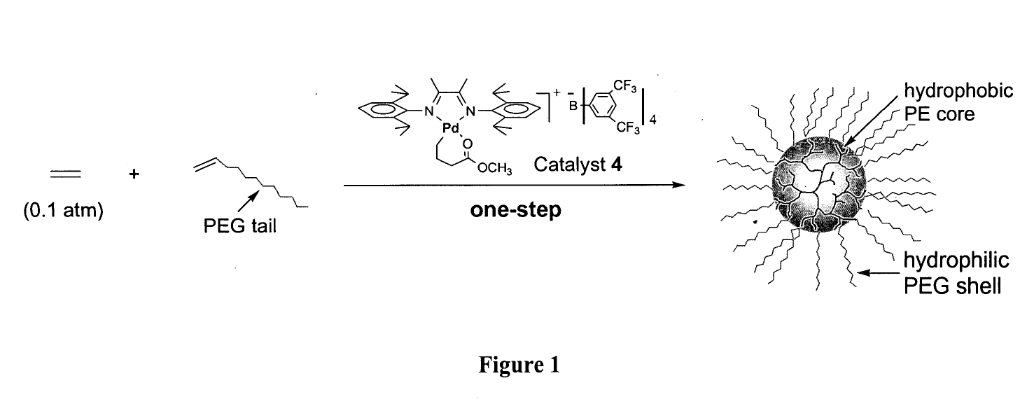

By copolymerizing ethylene with a comonomer having a poly(ethyleneglycol) (PEG) tail using the Brookhart palladium-α-diimine chain walking catalyst (Johnson, L. K.; Killian, C. M.; Brookhart, M. J. Am. Chem. Soc. 1995, 117, 6414-6415; Johnson, L. K.; Mecking, S.; Brookhart, M. J. Am. Chem. Soc. 1996, 118, 267-268) a core-shell dendritic polymer with a hydrophobic polyethylene (PE) core and a hydrophilic PEG shell was obtained in one step as depicted in FIG. 1.

We have shown previously that the branching topology of ethylene homo- and copolymers can be systematically tuned in a single synthetic operation by using the chain walking catalyst (Guan, Z.; Cotts, P. M.; McCord, E. F.; McLain, S. J. Science 1999, 283, 2059-2062; Guan, Z. Chem.-Eur. J. 2002, 8, 3086-3092; Guan, Z. J. Polym. Sci., Part A: Polym. Chem. 2003, 41, 3680-3692; Chen, G.; Ma, X. S.; Guan, Z. J. Am. Chem. Soc. 2003, 125, 6697-6704). Linear, hyperbranched, and dendritic copolymers containing a range of functionalities were obtained by changing ethylene pressures for copolymerizations (Chen, G.; Ma, X. S.; Guan, Z. J. Am. Chem. Soc. 2003, 125, 6697-6704). Because dendritic topology is obtained at low ethylene pressure and the insertion of substituted olefins occurs only when Pd walks to a primary carbon, that is, a chain end (Gottfried, A. C.; Brookhart, M. Macromolecules 2003, 36, 3085-3100.), copolymerization of ethylene and an PEG containing olefin at low ethylene pressure will produce a dendritic copolymer with a hydrophobic PE core and a hydrophilic PEG shell.

The synthesis of the water-soluble core-shell copolymers is shown in Scheme 5 below.

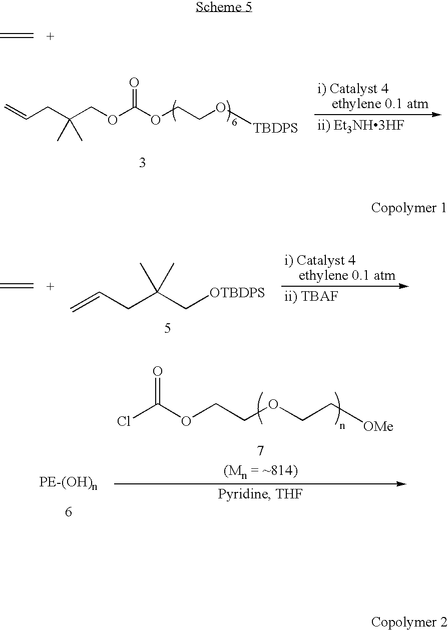

Comonomer 3 with a hexa(ethyleneglycol) tail was prepared by coupling a tert-butyldiphenylsilyl (TBDPS) protected mono-alcohol with 2,2-dimethyl-pent-4-enyl chlorofor-mate. Copolymerization of ethylene and 3 at 0.1 atm ethylene pressure followed by deprotection of TBDPS afforded copolymer 1 in one step. The number-averaged molecular weight (Mₙ) for copolymer 1 is 9800 g/mol as measured by size exclusion chromatography (SEC) coupled with a multi-angle light scattering (MALS) detector. The comonomer incorporation ratio (r) was determined to be 24 mol % by ¹H NMR. A much larger core-shell copolymer 2 was synthesized by a two-step approach. Here, an ethylene copolymer 6 having many hydroxyl groups was first prepared and subsequently coupled to PEG to afford the copolymer 2.

Copolymer 2 has a much higher molecular weight (Mₙ=463,000 g/mol, radius of gyration R_{g}=20.8 nm in THF) but similar comonomer incorporation ratio (r=26 mol %). Both copolymer 1 and 2 are soluble in water with solubility up to 10 g/L. They form stable molecular solution in water as evidenced by light scattering (LS) studies. The hydrodynamic radius (Rₕ) and R_{g} for the copolymer 2 in water were measured to be 26.0 and 17.7 nm, respectively. The slight decrease of R_{g} in water versus in THF was presumably due to contraction of the hydrophobic PE core in water. The ratio of Rₕ/R_{g} is around 1.4, which is close to the theoretical value of a filled sphere, 1.3 (Burchard, W. Adv. Polym. Sci. 1983, 48, 1-124). These data support that the copolymers exist as unimolecular nanospheres in water (See supporting information for detailed LS characterizations).

Nile Red, a common hydrophobic dye and an excellent UV/VIS and fluorescence probe (Watkins, D. M.; Sayed-Sweet, Y.; Klimash, J. W.; Turro, N. J.; Tomalia, D. A. Langmuir 1997, 13, 3136-3141), was chosen for investigating the unimolecular micellar properties of copolymer 1 and 2 in water. Nile Red is insoluble and does not fluoresce in water, but once it is encapsulated inside micelles, its aqueous solution starts to fluoresce. We monitored the fluorescence intensity as we added different amounts of copolymer 1 or 2 into an aqueous dispersion of Nile Red (16 mg/L). For comparison, a classical small molecule surfactant, sodium dodecyl sulfate (SDS) was also used to encapsulate the dye in water.

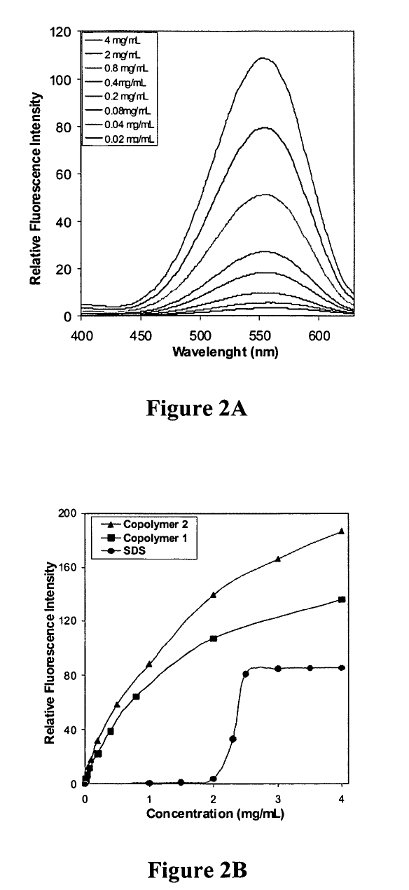

FIG. 2A shows that the fluorescence intensity of Nile Red increases gradually with the increase of copolymer 1 concentration, indicating copolymer 1 is effective in encapsulating Nile Red. The fluorescence intensities at λₘₐₓ of excitation spectra for Nile Red were plotted against the concentration of copolymer 1, copolymer 2 and SDS, respectively (FIG. 2B). For SDS, a typical S-shaped curve was obtained with the deflection point at its critical micelle concentration (CMC=2.3 mg/mL or 8 mM). At concentrations below its CMC, there is no micelle formation and SDS is unable to encapsulate Nile Red. There is a rapid increase in the number of SDS micelles above CMC, causing a sharp increase of encapsulated Nile Red. For the copolymer 1 and 2, on the contrary, the fluorescence intensity at λₘₐₓ for Nile Red increases gradually with the concentration of copolymers. No sharp transition was observed because there is no CMC for the covalently linked unimolecular nanocarriers.

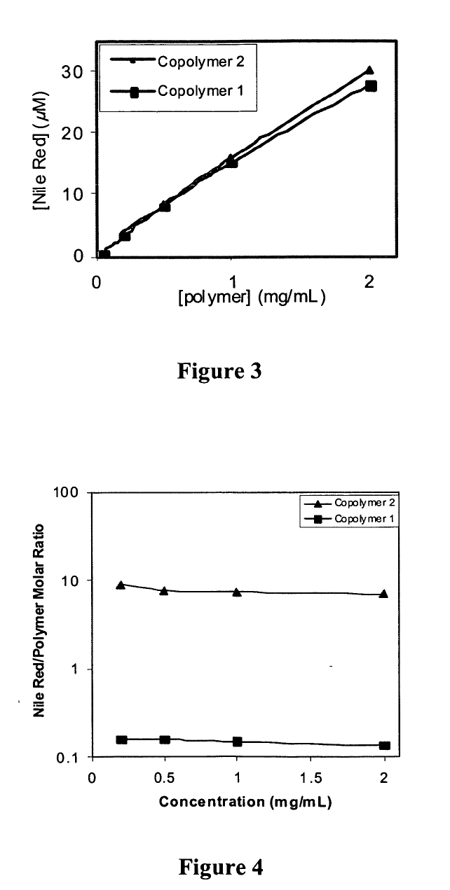

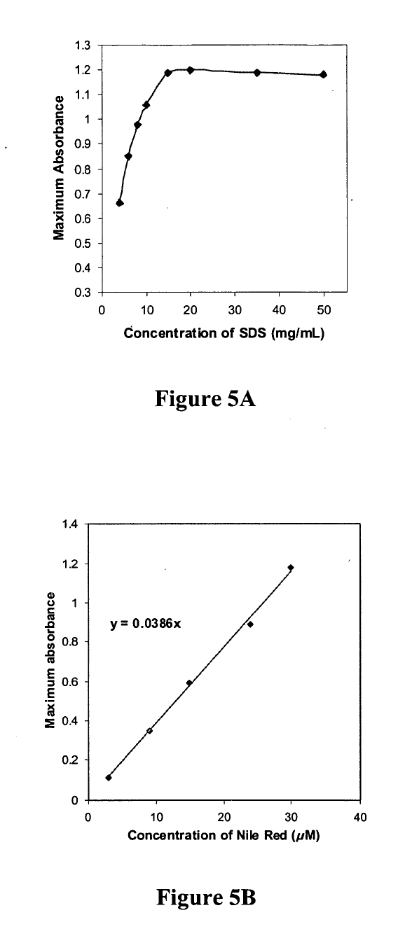

To further gain quantitative information of dye encapsulation, UV/Vis absorption experiments were conducted. The λₘₐₓ values for the UV is spectra of Nile Red are 554, 551, and 578 nm after addition of the copolymer 1, 2, and SDS, respectively. The blue shift in λₘₐₓ indicates the cores of our copolymers are more hydrophobic than that of the SDS micelle. The concentration of encapsulated Nile Red was obtained from its absorbance at λₘₐₓ using Beer's Law, which was plotted against the concentration of copolymer 1 or 2 (FIG. 3). The nearly linear increase of encapsulated dye with increasing concentration of copolymers indicates the copolymers behave as unimolecular micelles in water. The dye encapsulation capacities for copolymer 1 and 2 were quantified based on the UV/Vis data. The amounts of Nile Red being encapsulated by unit amount of copolymer 1 or 2 are nearly the same, that is, 16 μmol Nile Red per gram of copolymer or 0.46 wt %. This value is about twice as much as for SDS micelle (0.21 wt % at 4 mg/mL SDS). Copolymer 1 has a number-of-dye per polymer molecule of 0.15 while copolymer 2 shows a fifty-fold increase to 7.6. These values are constant at different copolymer concentrations ranging from 0.2 to 2 mg/mL for both copolymers (FIG. 4), indicating that they reflect the inherent property of the copolymers. Based on the molecular weights and chemical compositions, it is estimated that the Mₙ of the hydrophobic core of copolymer 2 (˜116,000 g/mol) is about 40 times as that of copolymer 1 (˜3,000 g/mol). The correlation between the number-of-dye per polymer molecule and the molecular structure of the copolymers indicates that the dye encapsulation capacity for our copolymer is mainly determined by the size of the hydrophobic core.

In summary, the inventor has shown the first example of transition metal catalyzed one-pot synthesis of water-soluble amphiphilic molecular nanocarriers behaving like unimolecular micelles. Using the chain walking catalyst, copolymerization of ethylene and comonomer 3 afforded, in one step, amphiphilic copolymer 1 having a hydrophobic core and a hydrophilic shell. The light aqueous solution showed unimolecular micellar properties for the copolymers. Quantitative data indicated that the dye encapsulation capacity is nearly proportional to the Mₙ of the hydrophobic core. The unimolecular micellar properties coupled with the good water solubility and biocompatibility of PEG make these molecular nanocarriers promising candidates for many applications including drug delivery and controlled drug release.

### Synthesis and Characterization of Comonomers and Copolymers

General Procedure. The catalyst synthesis and handling were carried out in Vacuum Atmosphere gloves box filled with nitrogen. All other moisture and air-sensitive reactions were carried out in flame-dried glassware using magnetic stirring under argon or nitrogen. Removal of organic solvents was accomplished by rotary evaporation and is referred to as concentrated in vacuo. Flash column chromatography was performed using forced flow on EM Science 230-400 mesh silica gel.

NMR spectra were recorded on Bruker DRX400, Bruker GN500, or Bruker Omega500 MHz FT-NMR instruments. Proton and carbon NMR spectra were recorded in ppm and were referenced to indicated solvents. NMR Data was reported as follows: chemical shift, multiplicity (s=singlet, d=doublet, t=triplet, q=quartet), coupling constant(s) in Hertz (Hz) and integration. Multiplets (m) were reported over the range (ppm) at which they appear at the indicated field strength. Elemental analysis was preformed by Atlantic Microlabs, Norcross, Ga. High resolution mass spectra (HR-MS) were recorded on Micromass LCT or Micromass Autospec.

Toluene, THF, diethyl ether and dichloromethane and other solvents were purified by passing through solvent purification columns following the method introduced by Grubbs (Pangbom, A. B.; Giardello, M. A.; Grubbs, R. H.; Rosen, R. K.; Timmers, F. J. Organometallics 1996, 15, 1518-1520). Ultrahigh purity (UHP) N₂ and ethylene were obtained from Airgas. The palladium bisimine catalyst used in our polymerization was synthesized by following literature report (Johnson, L. K.; Mecking, S.; Brookhart, M. J. Am. Chem. Soc. 1996, 118, 267-268; Mecking, S.; Johnson, L. K.; Wang, L.; Brookhart, M. J. Am. Chem. Soc. 1998, 120, 888-899).

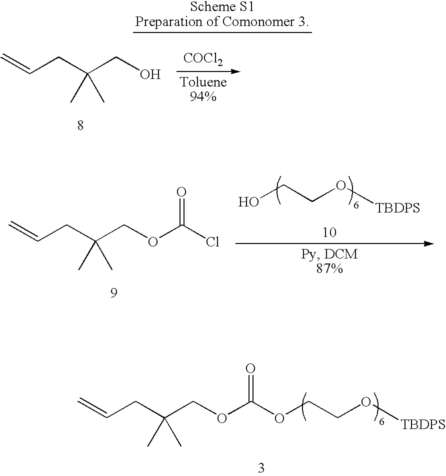

To a solution of 2,2-dimethyl-pent-4-enyl alcohol (Chen, G.; Ma, X. S.; Guan, Z. J. Am. Chem. Soc. 2003, 125, 6697-6704) (8, 2.28 g, 0.02 mol, 1.0 equiv.) in toluene (10 mL) was added phosgene solution in toluene (20 wt % or 1.93 M) (52 mL, 0.1 mol, 5 equiv.). The solution was allowed to stir for 24 hours. Solvent and excess phosgene was removed by concentrating in vacuo. Compound 2,2-dimethyl-pent-4-enyl chloroformate, 9, was obtained by drying the mixture under vacuum and used without further purification (3.32 g, 94%). At 0° C., a solution of 9 (1.94 g, 0.011 mol, 1.1 equiv.) in 20 mL DCM was slowly added to a solution of 17-(tert-butyldiphenylsiloxy)-1-hydroxy-3,6,9,12,15-pentaoxyheptadecane (Hawker, C. J.; Chu, F.; Pomery, P. J.; Hill, D. J. T. Macromolecules 1996, 29, 3831-3838), 10, (4.40 g, 0.01 mol, 1.0 equiv.) and pyridine (0.91 mL, 0.011 mol, 1.1 equiv.) in 50 mL DCM. The mixture was then allowed to stir at room temperature overnight. Aqueous solution of NH₄Cl was added to quench the reaction. Aqueous layer was separated and washed with DCM three times. Combined organic layers were washed with brine, dried over Na₂SO₄ and concentrated in vacuo. The pure product 3 was obtained in 87% yield after flash column chromatography (EtOAc in Hex: 0 to 20%). ¹H NMR (500 MHz, CDCl₃): δ 7.68-7.70 (m, 4H), 7.36-7.44 (m, 6H), 5.78-5.86 (m, 1H), 4.99-5.08 (m, 2H), 4.26-4.28 (m, 2H), 3.86 (s, 2H), 3.81 (dd, J₁=J₂=5.4, 2H), 3.60-3.70 (m, 20H), 2.04 (m, 2H), 1.05 (s, 9H), 0.093 (s, 6H). ³C NMR (125 MHz, CDCl₃): δ 155.61, 135.78, 134.38, 133.89, 129.77, 127.80, 118.03, 75.87, 72.61, 70.85 (m), 69.14, 67.04, 63.61, 43.38, 34.41, 27.00, 24.12, 19.37. HR-MS: calcd. for [C₃₆H₅₆O₉Si+H⁺]: 661.3694, found: 661.3731.

### Comonomer 5 was Prepared by Following Previous Report (Chen, G.; Ma, X S.; Guan, Z. J. Am. Chem. Soc. 2003, 125, 6697-6704).

General Procedure of Polymerization: Ethylene copolymerization with polar comonomers was performed in a 100 mL flame dried flask connecting to a bubbler filled with mineral oil. Low ethylene pressure (0.1 atm) was maintained by slowly bubbling a mixed gas of 90% N₂ and 10% ethylene at 1.0 atm. A degassed comonomer solution in chlorobenzene/toluene (1:3, by volume) was added into the flask followed by a degassed catalyst solution in chlorobenzene/toluene (1:3, by volume). After purged the flask for a few minutes with the polymerization gas, the N₂/ethylene gas mixture was bubbled constantly through the polymerization solution. Polymerization was allowed to continue for 24 to 48 hours at room temperature. The rate of the gas mixture flow was kept at about two bubbles per second as indicated by the bubbler. Polymerization was finally quenched by adding excess amount of triethylsilane. The resulting polymer solution was passed through Celite and neutral alumina gel to remove catalyst residue before being concentrated.

### Synthesis of Copolymer 1

The comonomer 3 (3.2 g, 0.0048 mol, 0.4 M) was copolymerized with ethylene at 0.1 atm with the catalyst 4 (100 mg, 0.07 mmol) for 48 hours. After addition of Et₃N•3HF (0.81 mL, 0.005 mol) at the end of polymerization, the solution was allowed to stir at room temperature for an additional 48 hours. The mixture was diluted with large amount of CHCl₃, which was washed with brine. The organic layer was separated and concentrated. The residue was then re-dissolved in a small amount of acetone. Pure copolymer 1 (0.7 g) was obtained by repetitive precipitation of the polymer from the acetone solution by addition of a mixture of hexanes and ethyl acetate (10:1). ¹H NMR (500 MHz, CDCl₃): δ 4.26 (s, 2H), 3.83 (m, 2H), 3.50-3.75 (m, 20H), 2.98 (broad, 1H), 1.02-1.35 (broad, 16.3H), 0.85-0.95 (broad, 7.0H). Comonomer incorporation ratio (r) is determined to be 24 mol % by ¹H NMR. Molecular weight of hydrophobic core (MW_{core}) is 3,000 g/mol. GPC-MALS (using THF as eluent): Mₙ=9,800±40 g/mol, Mₙ=13,100±500 g/mol, PDI=1.3.

### Synthesis of Copolymer 2

Comonomer 5, tert-butyl-(2,2-dimethyl-pent-4-enyloxy)-diphenyl-silane (Chen, G.; Ma, X. S.; Guan, Z. J. Am. Chem. Soc. 2003, 125, 6697-6704) (12.0 g, 0.034 mol, 1.0 M), was first copolymerized with ethylene at 0.1 atm with catalyst 4 (100 mg, 0.07 mmol) for 48 hours. After quenching the copolymerization, an excess amount of TBAF was added to the polymerization solution, which was allowed to stir at room temperature for an additional 24 hours. The hydroxyl functionalized polyethylene, 6, was obtained by repetitive precipitation of the polymer solution with methanol. It was then dried under vacuum, and re-dissolved in THF as a stock solution. Poly(ethyleneglycol) chloroformate (Ekwuribe, N. N.; Price, C. H.; Ansari, A. M.; Odenbaugh, A. L. In PCT Int. Appl.; (Nobex Corporation, USA). Wo, 2002, p 201 pp) (7, 2.4 g, 0.003 mol, Mₙ=˜814) was added into the above polymer 6 solution (30 mL, 0.6 g of 6) in the presence of pyridine (0.24 mL, 0.003 mol). The reaction mixture was allowed to stir at room temperature for 24 hours before being diluted with a large volume of CHCl₃ and washed with brine. The organic layer was separated and concentrated. The residue was then re-dissolved by small amount of acetone. Pure copolymer 2 was obtained in 83% yield by repetitive precipitation of the polymer from the acetone solution by addition of a mixture of hexanes and ethyl acetate (10:1). ¹H NMR (500 MHz, CDCl₃): δ 4.25 (broad, 4H), 3.50-3.84 (broad, 64H), 3.38 (s, 3H), 0.95-1.35 (broad, 15.8H), 0.85-0.95 (broad, 6.6H). PEG incorporation ratio (r) is determined to be 26 mol % by ¹H NMR. Molecular weight of hydrophobic core (MW_{core}) is ˜116,000 g/mol. GPC-MALS (using THF as eluent): Mₙ=463,000±2,800 g/mol, Mw=631,300±2,500 g/mol, PDI=1.4, radius of gyration R_{g}=20.8±0.8 nm.

### SEC-MALS Characterization of the Copolymers in THF

All of the above polymers were characterized by size-exclusion chromatography (SEC) coupled to a multi-angle light scattering detector (MALS) for obtaining both the molecular weight (M) and the radius of gyration (R_{g}), substantially following protocols previously reported (Guan, Z.; Cotts, P. M.; McCord, E. F.; McLain, S. J. Science 1999, 283, 2059-2062; Cotts, P. M.; Guan, Z.; McCord, E.; McLain, S. Macromolecules 2000, 33, 6945-6952). Measurements were made on highly dilute fractions eluting from a SEC consisting of a HP Agilent 1100 solvent delivery system/auto injector with an online solvent degasser, temperature-controlled column compartment, and an Agilent 1100 differential refractometer. A Dawn DSP 18-angle light scattering detector (laser wavelength λ=632 nm, Wyatt Technology, Santa Barbara, Calif.) was coupled to the SEC to measure both the molecular weights and sizes for each fraction of the polymer eluted from the SEC column. A 30 cm column was used (Polymer Laboratories PLgel Mixed C, 5 μm particle size) to separate polymer samples. The mobile phase was THF and the flow rate was 0.5 mL/min. Both the column and the differential refractometer were held at 35° C. A 60 μL of a 2 mg/mL solution was injected into the column. Software ASTRA 4.7 from Wyatt Technology was used to acquire data from the 18 scattering angle detectors and the differential refractometer. The M_{w}, Mₙ, R_{g} data were obtained by following classical light scattering treatments. The R_{g} data reported are the weight-averaged values.

### Characterization of the Unimolecular Properties of Copolymer 2 in Water by Static and Dynamic Light Scattering

Both dynamic and static light scattering measurements of copolymer 2 aqueous solutions were performed at the Wyatt Technology Corporation, Santa Barbara, Calif. A QELS detector was coupled to a Dawn EOS 18-angle light scattering detector (laser wavelength λ=690 nm) for dynamic light scattering information. The measurements were done using batch mode in pure water at 25° C. Software ASTRA 4.90 from Wyatt Technology was used to acquire data from the 18 scattering angle detectors and QELS.

Copolymer 2 only: Static light scattering: the weight-average radius of gyration, R_{g}, is 17.7±4.0 nm. Dynamic light scattering: the average hydrodynamic radius, Rₕ, is 26.0±0.6 nm. Measurements for dynamic light scattering were done at copolymer 2 concentration of 1.0 mg/mL.

Copolymer 2 with Nile Red: The sample of copolymer 2 with Nile Red was prepared by adding copolymer 2 into 10 mL of the prepared Nile Red aqueous dispersion (16 mg/L). The mixture was sonicated for a few hours at room temperature to allow for reaching equilibrium.

Static light scattering: R_{g} of copolymer 2 with Nile Red encapsulated in water is 17.9±4.0 nm. Dynamic light scattering: Rₕ of copolymer 2 with Nile Red encapsulated in water is 26.2±0.6 nm. Measurements for dynamic light scattering were done at copolymer 2 concentration of 1.0 mg/mL.

### Fluorescence and UV/Vis Spectroscopic Studies of the Copolymers

Nile Red (also known as Nile Blue A Oxazone, or NBAO) was chosen as the hydrophobic dye for the encapsulation studies (Watkins, D. M.; Sayed-Sweet, Y.; Klimash, J. W.; Turro, N. J.; Tomalia, D. A. Langmuir 1997, 13, 3136-3141). To determine the dye encapsulation capacities of the copolymers, an aqueous dispersion of Nile Red (16 mg/L) was prepared through sonication. Samples for UV/Vis and fluorescence spectroscopic studies were prepared by adding copolymer 1, copolymer 2, or sodium dodecyl sulfate (SDS, 99%, Acros) into 5 mL of the prepared Nile Red aqueous dispersion. The mixtures were sonicated for a few hours at room temperature to allow reaching equilibrium. After being filtered through 0.2 μm hydrophilic filter (Whatman), the solutions were clear and stable for months.

All fluorescence spectra were recorded on a JASCO FP-750 fluorescence spectrophotometer, which used a 150-watt Xenon lamp with shielded lamp house as light source. Emission spectra was obtained using excitation wavelength at 570 nm for all of the samples. Excitation spectra was obtained using emission wavelength at 650 nm for all of the samples. The λₘₐₓ values for emission fluorescence spectra of Nile Red dye are 626 nm, 621 nm and 643 nm after addition of copolymer 1, copolymer 2 and SDS, respectively (FIGS. 2A and 2B). The λₘₐₓ values for excitation fluorescence spectra of Nile Red dye are 556 nm, 552 nm and 578 nm after addition of copolymer 1, copolymer 2 and SDS, respectively.

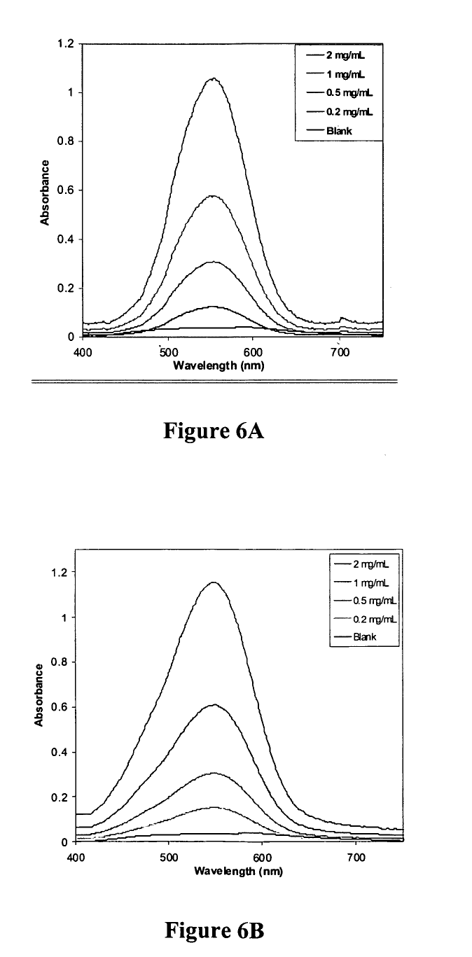

All UV/Vis spectra were recorded on a JASCO V-530 UV/V is dual-beam spectrophotometer, which used a deuterium lamp as light source for the UV range (190-350 nm) and a halogen lamp for the visible light range (330-1100 nm). The concentrations of Nile Red encapsulated by micelles are calculated from Beer's Law: A=cεl; Where ε is the molar extinction coefficiency, c is the molar concentration of the encapsulated dye, and l is the path length of the sample cell (cm). The E value of Nile Red is nearly constant in most organic solvents with a value of 38,000 M⁻¹ cm⁻¹. No E data is available for Nile Red in aqueous media because of its water insolubility. We chose SDS as the model system to determine the ε value of Nile Red encapsulated by micelles in water. For this purpose, SDS was gradually added to a Nile Red suspension (9.55 mg/L, or 30 μM) until the saturation of the absorbance was observed in the resulting solutions (FIG. 5A). A constant absorbance was observed at the end of the curve because all of the dye molecules were encapsulated after the saturation point. This measurement was repeated for a range of Nile Red concentrations (3-30 EM). As shown in FIG. 5B, maximum absorbance of these Nile Red aqueous solutions followed linear relationship with Nile Red concentration. The slop of this curve gave the ε of Nile Red in aqueous micelle solutions as 38,6000±800 M⁻¹ cm⁻¹. This value was used in our study of Nile Red encapsulation by SDS, copolymer 1 and 2 unimolecular micelles.

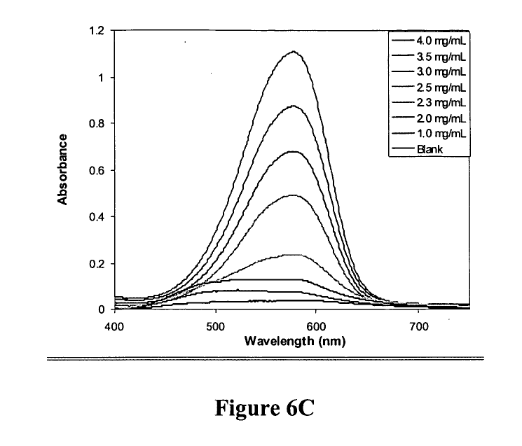

The λₘₐₓ values for the UV/Vis spectra of Nile Red dye are 554 nm, 551 nm and 578 nm after addition of copolymer 1, copolymer 2 and SDS, respectively (FIGS. 6A, 6B, and 6C). The average number-of-dye per polymer molecule (dye/polymer molar ratio) was calculated from the ratio of their concentrations. The dye encapsulation capacities in weight percentage (dye/polymer weight ratio that reported in percentage) were calculated from the dye/polymer molar ratios and their molecular masses.

TABLE S1
Quantitative Dye Encapsulation Capacity Data of Copolymers and SDS.

| COPOLYMER 1 | | | COPOLYMER 2 | | | SDS | |
|---|---|---|---|---|---|---|---|
| | DYE/POLYMER RATIO | | | DYE/POLYMER RATIO | | | DYE/SDS |
| CONC. | Molar Ratio | Weight Ratio^{c} | CONC. | Molar Ratio | Weight Ratio^{c} | CONC. | WEIGHT RATIO |
| 0.2 | 0.156 | 0.459% | 0.2 | 8.74 | 0.525% | 2.5 | 0.077% |
| 0.5 | 0.156 | 0.459% | 0.5 | 7.57 | 0.472% | 3.0 | 0.147% |
| 1.0 | 0.146 | 0.429% | 1.0 | 7.28 | 0.454% | 3.5 | 0.169% |
| 2.0 | 0.134 | 0.398% | 2.0 | 6.91 | 0.431% | 4.0 | 0.207% |
| AVG^{B} | 0.15 | 0.44% | AVG^{B} | 7.6 | 0.47% | N/A | N/A |

Note:
(a): mg/mL;
^{B}average of values with different concentrations of copolymer 1 or copolymer 2 (not applicable to SDS);
^{c}dye/polymer weight ratio was reported in percentage.

Thus, specific embodiments and applications of transition metal-catalyzed syntheses of dendritic polymers have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Furthermore, where a definition or use of a term in a reference, which is incorporated by reference herein is inconsistent or contrary to the definition of that term provided herein, the definition of that term provided herein applies and the definition of that term in the reference does not apply.

What is claimed is:

1. An amphiphilic core-shell copolymer having a core with a plurality of branches, wherein the branches have unequal distances between at least two branch points, wherein the core comprises a first polymer, wherein the copolymer further comprises a shell that comprises a second polymer, and wherein the second polymer is covalently coupled to a terminus of a branch of the first polymer.

2. The core-shell copolymer of claim 1 wherein the first polymer has a dendritic structure.

3. The core-shell copolymer of claim 1 wherein the first polymer is hydrophobic and wherein the shell polymer is hydrophilic.

4. The core-shell copolymer of claim 1 wherein the first polymer is fluorophobic and wherein the shell polymer is fluorophilic.

5. The core-shell copolymer of claim 1 wherein the first polymer comprises a polyolefin and wherein the second polymer comprises a polyethylene glycol.

6. The core-shell copolymer of claim 1 wherein the second polymer further comprises a reactive group suitable for derivatization with a biological molecule.

7. A reaction mixture comprising a plurality of first monomers and second monomers, and a polymerization catalyst capable of a chain walking reaction, wherein the second monomer is functionalized with a group such that (a) the second monomer is hydrophilic, or that (b) the group is suitable for reaction with a hydrophilic reagent.

8. The reaction mixture of claim 7 wherein the first and second monomers comprise an optionally substituted ethylene group.

9. The reaction mixture of claim 8 wherein the first monomers comprise an α-olefin and wherein the second monomers comprise a 2,2-dimethyl-pent-4-enyl-epoxide, an optionally protected 2,2-dimethyl-pent-4-enyl-alcohol or an optionally protected 2,2-dimethyl-pent-4-enyl-acid.

10. The reaction mixture of claim 7 wherein the polymerization catalyst comprises an organometallic catalyst.

11. The reaction mixture of claim 10 wherein the polymerization catalyst comprises a late transition metal in complex with at least one coordinating atom.

12. The reaction mixture of claim 10 wherein the second monomer is hydrophilic and further includes a reactive group suitable for derivatization with a biological molecule.

13. A method of forming a dendritic amphiphilic polymer, comprising:
providing a plurality of first monomers and a plurality of second monomers, wherein at least some of the second monomers include a hydrophilic group or a group suitable for reaction with a hydrophilic reagent; and
reacting the first and second monomers under conditions that promote (a) formation of a branched polymer, and (b) covalent bonding of the second monomers to termini of branches of the branched polymer.

14. The method of claim 14 wherein the dendritic amphiphilic polymer has a hydrophobic core and a hydrophilic shell.

15. The method of claim 13 wherein the first monomers have a structure of R₁R₂C═CR₃R₄, wherein R₁, R₂, R₃, and R₄ are independently hydrogen, halogen, and optionally substituted lower alkyl.

16. The method of claim 13 wherein the second monomers have a structure of R₁R₂C═CR₃R₅, wherein R₁, R₂, and R₃, are independently hydrogen, halogen, and optionally substituted lower alkyl, and wherein R₅ comprises a polar group.

17. The method of claim 16 wherein the polar group is selected from the group consisting of a hydroxy group, a carboxy group, an epoxy group, a substituted ester, a substituted amide, a substituted imide, a polyol, and a polyether.

18. The method of claim 13 wherein the group suitable for reaction with the hydrophilic reagent is an alcohol, an acid, or an epoxy group, and further comprising a step of reacting the alcohol, acid, or epoxy group with a polar reagent.

19. The method of claim 13 wherein the step of reacting comprises a step of chain walking polymerization.

20. The method of claim 13 wherein the step of chain walking polymerization is performed at a pressure of less than 0.5 atm of the first monomer.
